(19) United States
(12) Patent Application Publication
(10) Pub. No.: US 2011/0091571 A1
(43) Pub. Date: Apr. 21, 2011

(54) METHOD OF CONTROLLING THE PROPAGATION OF MRSA, STAPH AND OTHER INFECTIONS THAT COLONIZE IN THE NOSE

(76) Inventor: Michael F. Moore, Dunmore, PA (US)

(21) Appl. No.: 12/906,493

(22) Filed: Oct. 18, 2010

Related U.S. Application Data
(60) Provisional application No. 61/252,597, filed on Oct. 16, 2009.

(51) Int. Cl. A61K 33/38; A61P 31/04

(52) U.S. Cl. 424/618

(57) ABSTRACT

A method or protocol for reducing the spread of bacterial infections such as MRSA among a population includes identifying a group of individuals at risk of carrying or becoming carriers of a bacterial infection that colonizes in the nose, determining whether any individuals can be excluded from treatment due to reasons relating to the individual's other activities or precautions taken, designating an individual or group of individuals who will receive treatment, providing a strip, the strip having an effective amount of antimicrobial agent capable of being delivered transdermally, the strip is exteriorly applied to the nose of the designated individual or group of individuals so to allow the antimicrobial agent to pass through the nose to interact with the colony to suppress the colonization, or altogether eradicate the colony.

### CROSS REFERENCE TO RELATED APPLICATIONS

This application claims priority to U.S. provisional patent application entitled METHOD OF CONTROLLING THE PROPAGATION OF MRSA, STAPH AND OTHER INFECTIONS THAT COLONIZE IN THE NOSE, No. 61/252,597 filed on Oct. 16, 2009.

### STATEMENT AS TO FEDERALLY SPONSORED RESEARCH

Not applicable.

### BACKGROUND OF INVENTION

a. Field of Invention

The invention relates generally to methods for controlling the spread of bacterial infections within health care facilities, skilled nursing facilities, institutional care facilities such as prisons, dormitories, military barracks, or anywhere people come in close proximity—increasing the incidence of the spread of infection among a population. Particularly, the invention relates to a method or protocol for reducing the spread of infection in the abovementioned situations by providing an adhesive strip metalized with silver or other effective antimicrobial agent and applying the strip to the nose for transdermal delivery to eradicate the colonization of bacteria.

b. Background of Invention

Methicillin-resistant staphylococcus aureus (MRSA) is a bacterium responsible for difficult-to-treat infections in humans. It may also be referred to as multidrug-resistant staphylococcus aureus or oxacillin-resistant staphylococcus aureus (ORSA). MRSA is by definition a strain of Staphylococcus aureus that is resistant to a large group of antibiotics called the beta-lactams, which include the penicillins and the cephalosporins. The invention is primarily targeted at controlling the spread of MRS A, but is also intended at being effective at other bacterium that colonize in the nose.

MRSA is a resistant variation of the common bacterium Staphylococcus aureus. It has evolved an ability to survive treatment with beta-lactam antibiotics, including methicillin, dicloxacillin, nafcillin, and oxacillin. MRSA is especially troublesome in hospital-associated (nosocomial) infections. In hospitals, patients with open wounds, invasive devices, and weakened immune systems are at greater risk for infection than the general public. The infection may spread in a number of ways. A patient may come to a health care facility having the infection. Since the infection colonizes in the nose, a patient may easily spread the infection by touching or wiping the nose and any other article. Hospital staff who do not follow proper sanitary procedures may transfer bacteria from patient to patient. Visitors to patients with MRSA infections or MRSA colonization are advised to follow hospital isolation protocol by using the provided gloves, gowns, and masks if indicated. Visitors who do not follow such protocols are capable of spreading the bacteria to cafeterias, bathrooms, and elevators.

The organism is often sub-categorized as community-acquired MRSA (CA-MRSA) (community acquired is also associated with a toxin PVL toxin) or health care-associated MRSA (HA-MRSA) (It is also known as Hospital acquired MRSA) although this distinction is complex. Some have defined CA-MRSA by characteristics of patients who develop an MRSA infection while other authors have defined CA-MRSA by genetic characteristics of the bacteria themselves. The first reported cases of community-acquired MRSA began to appear in the mid-1990s from Australia, New Zealand, the United States, the United Kingdom, France, Finland, Canada, and Samoa, notable because they involved people who had not been exposed to a health-care setting. The new CA-MRSA strains have rapidly become the most common cause of cultured skin infections among individuals seeking emergency medical care in urban areas of the United States. These strains also commonly cause skin infections in men who have sex with men, athletes, prisoners and soldiers.

There are two major ways people become infected with MRSA. The first is physical contact with someone who is either infected or is a carrier (people who are not infected but are colonized with the bacteria on their body) of MRSA. The second way is for people to physically contact MRSA on any objects such as door handles, floors, sinks, or towels that have been touched by an MRSA-infected person or carrier. It is known that MRSA colonizes in the nose, and thus in health care settings, patients are sometimes screened for MRSA by taking a culture of the nose. If positive, the patient is treated with nose drops having an effective antimicrobial agent, and subsequent cultures are taken to determine of the infection is eradicated. If the patient tests positive, surgical procedures are postponed.

b. Description of Related Art

The following patents are representative of the field pertaining to the present invention:

U.S. Pat. No. 7,087,249 describes the use of one or more antimicrobial metals preferably selected from silver, gold, platinum, and palladium but most preferably silver, formed with atomic disorder, and preferably in a nanocrystalline form, for reducing inflammation or infection of the mucosal membrane. Transdermal patches may provide controlled delivery of the antimicrobial metal to the mucosal membrane. For example, an adhesive patch or adhesive matrix patch, can be prepared from a backing material and an adhesive, such as an acrylate adhesive. Powders or solutions of the antimicrobial metal may be formulated into the adhesive casting solution and allowed to mix thoroughly. The solution is cast directly onto the backing material and the casting solvent is evaporated in an oven, leaving an adhesive film. Alternatively, a poly-urethane matrix patch can be employed to deliver the antimicrobial metal to the mucosal membrane. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and drug to the prepolymer results in the formation of a tacky firm elastomer that can be directly cast onto the backing material.

U.S. patent application Ser. No. 11/101,386 discusses method of preventing or treating staphylococcal bacterial infection in an individual is disclosed. A vaccine based on a conjugate the 336 polysaccharide antigen can be used for active protection in individuals who are to be subjected to conditions that place them at immediate risk of developing a bacterial infection, as would be case in the context of a catheterization or a surgical procedure. Alternatively, antibodies raised in response to the antigen can be used to treat or to provide passive protection to individuals. The method can be used in a population of patients at risk for infection by various species of Staphylococcus or various types of Staphylococcus aureus.

U.S. patent application Ser. No. 11/490,512 discloses a vaccine based on a conjugate of PS1 polysaccharide antigen can be used for active protection in individuals who are to be subjected to conditions that place them at immediate risk of developing a bacterial infection, as would be case in the context of a catheterization or a surgical procedure. Alternatively, antibodies raised in response to the antigen can be used to treat or to provide passive protection to individuals. The method can be used in a population of patients at risk for infection by various species of Staphylococcus or various types of Staphylococcus epidermidis.

U.S. patent application Ser. No. 11/432,876 describes a compression stretch bandage formed from at least one layer of a stretchable, textile material forming a body of the bandage, a base material attached to the stretchable, textile material on a first side, and a silver material attached to the base material for reducing risk of infection. The bandage may be a flexible, stretchable, hydrophilic bandage that reduce the risk of infection at a wound by providing a moist environment that will aid in optimum release of silver ions into the wound.

U.S. patent application Ser. No. 10/836,530 describes a method for enhancing the metal ion release rate of a substrate having a coating of a metal thereon. The method includes the steps of forming the metal-coated substrate and then subjecting the metal-coated substrate to a step that removes portions of the metal coating to form at least one notch in the metal coating, thereby increasing the surface area of the metal coating. The increased surface area enhances the metal ion release rate of the substrate. The metal may be silver. A silver-coated substrate may be used in the formation of medical products having increased antimicrobial and/or anti-fungal characteristics.

The prior art Breathe Rite® nose strips consist of a spring member strip with an adhesive backing. The device enlarges the nasal passageways through the affixing of the flexible adhesive strip, low across the bridge of the nose.

Notwithstanding the prior art, the present invention is neither taught nor rendered obvious thereby.

### SUMMARY OF INVENTION

The invention relates to a method or protocol for reducing the spread of bacterial infections such as MRSA among a population.

In a preferred embodiment of the present invention, the method includes identifying a group of individuals at risk of carrying or becoming carriers of a bacterial infection that colonizes in the nose. Identification step may alternately include identifying an activity or location of an individual that puts said individual at risk of carrying the infection or becoming a carrier of said infection.

Once the identification is made, the next step may involve determining whether any individuals can be excluded from treatment due to reasons relating to the individual's other activities or precautions taken, which may result in the individual having a lower risk of carrying or becoming a carrier relative to the remaining members of the classification.

The next step involves designating an individual or group of individuals who will receive treatment with the strip based on the identification/exclusion steps above. In some embodiments, treatment is prophylactic in nature, wherein the designated individual or group of individuals is/are not confirmed to be carriers of a bacterial infection that colonizes in the nose.

The next step of the invention includes providing a strip, the strip having an effective amount of antimicrobial agent capable of being delivered transdermally. Preferably, the antimicrobial agent comprises silver. However, any agent capable of eradicating the bacterial colonization of the nose is suitable for use with the strip.

Next, the strip is exteriorly applied to the nose of the designated individual or group of individuals so to allow the antimicrobial agent to pass through the nose to interact with the colony to suppress the colonization, or altogether eradicate the colony.

In some preferred embodiments of the present invention, the designated individual or group of individuals may be cultured to determine the effectiveness of the treatment, or otherwise determine whether the bacterial infection continues to exist.

In some preferred embodiments of the present invention, the application of the strip may be in addition to other measures, procedures, or treatments directed at suppressing, mediating, eradicating, or reducing the spread of the bacterial infection among a population.

Additional features, advantages, and embodiments of the invention may be set forth or apparent from consideration of the following detailed description. Moreover, it is to be understood that both the foregoing summary of the invention and the following detailed description are exemplary and intended to provide further explanation without limiting the scope of the invention.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 is a schematic representation of a preferred embodiment of the method;

FIG. 2 is a schematic diagram of a preferred embodiment of the identification/exclusion/classification step of the method;

FIG. 3 is a schematic diagram of embodiments of the transdermal antimicrobial strip used in the method;

### DETAILED DESCRIPTION OF THE EMBODIMENTS

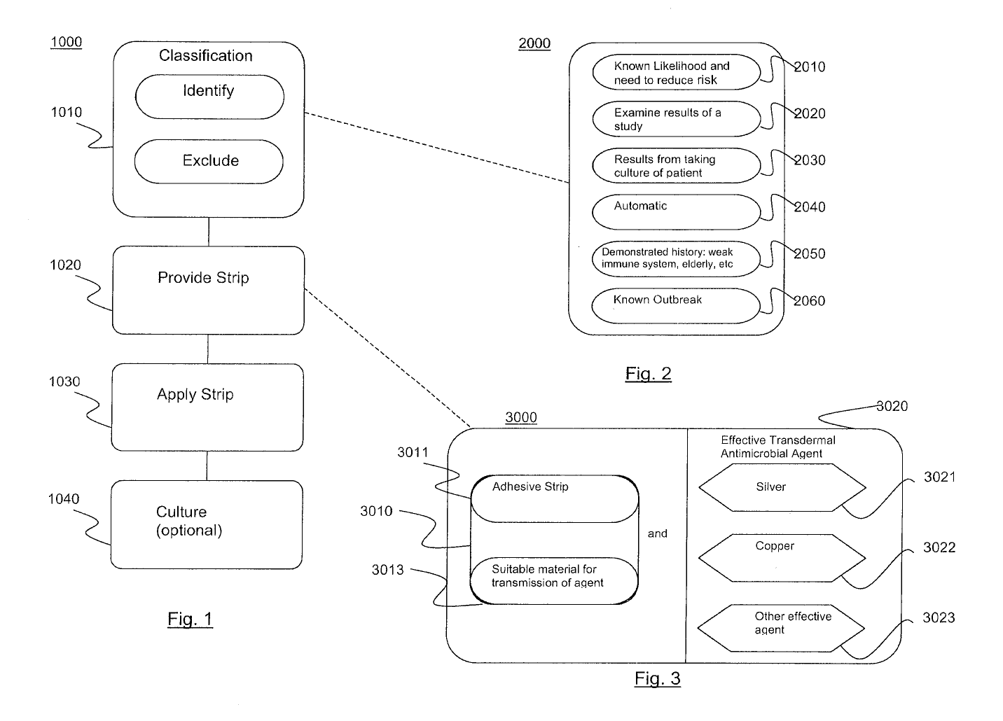

Referring to FIG. 1, the invention relates to a method for controlling the spread of bacterial infections within health care facilities, skilled nursing facilities and, institutional care facilities such as prisons, dormitories, military barracks, or anywhere people come in close proximity—increasing the incidence of the spread of infection among a population. Particularly, the invention concerns a method or protocol 1000 for reducing the spread of infection in the abovementioned situations by providing an adhesive strip containing silver or an other effective antimicrobial agent and applying the strip to the nose for transdermal delivery to eradicate or inhibit the colonization of bacteria.

In a preferred embodiment of the present invention, the method includes step 1010 identifying a group of individuals who are carriers, or at risk of carrying or becoming carriers, of a bacterial infection that colonizes in the nose. Identification step 1010 may alternately include identifying an activity or location of an individual that puts said individual at risk of carrying the infection or becoming a carrier of said infection.

Referring to FIG. 2, exemplary identification sub-steps may include, identification from a known likelihood of incidence of a bacterial infection that colonizes in the nose, and a need to reduce the risk of contracting the infection 2010. Examples include nosocomial infections occurring at hospitals, skilled nursing facilities, or other health care institutions. Anywhere people come in close proximity generally increases the risk of spreading a bacterial infection. This includes dorms, military barracks, and camps. Areas of confined containment such as in planes, trains, and subways are identifiable risks for spreading the bacterial infection.

Similarly, identification may result from conducting a study, or examining the results of a study 2020 on incidences of bacterial infection propagation among a population. Examples include published or unpublished reports showing populations, activities, and/or locations that have a high incidence of infection resulting from colonization of the noses of carriers.

Direct identification is accomplished by taking cultures (step 2030) from patients or other individuals to determine whether they are infected or carriers of the targeted bacterial infection. Cultures may be ordered as a preoperative care to identify carriers. Healthcare workers and the family members of carriers may also be screened for MRSA or other bacterial infection. When an outbreak occurs, numerous screens may be performed to help identify the source of the infection. In some settings, such as nursing homes, a large number of people may be screened to evaluate the spread of colonization in a specific population.

In certain circumstances, identification of individuals, a group, or population for treatment with the strip is made automatically (step 2040), as part of a prophylactic approach to minimizing the likelihood of colonization. For example, newly admitted patients, new residents of nursing homes, care facilities, and patients receiving preoperative care, receive treatment with the strip without any previous cultures. Similarly, if the infection is found to be present among a population, automatic application to all patients (or members or classes of the population) may take place.

In other instances, identification is based on a demonstrated history of a population, group, or activity that results in a high incidence of infection and/or colonization (step 2050). For example, contact activities such a football or wrestling may have a demonstrated history of a high risk of transmitting MRSA, accordingly prophylactic treatment may be applied to these groups/activities.

In other instances, a known outbreak among a population or group, or individuals associated with a particular activity (step 2060) will lead to identification of candidates for treatment.

Referring to FIG. 3, there is shown a schematic relating to the transdermal antibiotic strip 3000 of the method 1000. Preferably, the strip is adhesive on at least one surface 3011 and is made of a material suitable for transmission 3013 of the transdermal antimicrobial agent 3020. Preferably, the agent 3020 comprises silver 3021. Alternately, the agent 3020 comprises copper 3022 or another transdermal antimicrobial agent 3023 capable of suppressing or eradicating the colonization of the targeted bacteria.

Silver—In a preferred embodiment, the transdermal antimicrobial agent 3020 used for delivery by the strip 3000 contains silver 3021. The strip may contain a plurality of layers having a silver coating 3021, or a compound containing silver 3021, which reacts with skin or moisture to release silver to the colony in the nose. Alternately at least one layer of the strip 3000 is impregnated with a silver containing compound 3021 capable of releasing silver ions when the strip is in contact with skin.

Alternate antimicrobial agents include copper 3022 or any other known antimicrobial agent 3023 capable of transdermal delivery from the strip 3000.

After the classification/identification of the population or group of individuals, application of the strip step 1030 is undertaken. Strips 1020 are applied to the outside of the nose of those individuals identified in the classification. As discussed herein, in healthcare settings, the strips are applied to newly admitted patients, individuals who are admitted to elder care facilities, patients with weakened immune systems, patients receiving preoperative treatment, visitors, and possibly staff. In other settings, application of the strip is in reaction to an outbreak associated with a location or activity. In other settings, the strip is applied to individuals associated with an at risk activity such as being located in dense populations, or activities taking place in confined spaces, or where individuals are in close contact or proximity. Examples include traveling in trains, planes, subways, living in close quarters such as dorms, clinics, hospitals, military barracks, schools child care situations, and elder care situations. Other examples include sports activities where players are in close contact, such as basketball, football, and wrestling.

In some embodiments of the method, follow up screening of individuals is conducted by taking cultures of the nose, or other sites where infection is suspected. Follow up screening may also include other procedures known in the art at identifying symptoms associated with an active infection.

The invention may further include the step of providing MRSA education and training information to assist responsible individuals in identifying outbreaks and infection risks and undertaking a regimen, which may include the application of the strips 1030. For example, in a university setting, university administrators are provided with education and training concerning the actual or perceived infection (MRSA, for example), how to identify, how to minimize an outbreak with proper sanitation procedures, how and when to use the inventive strip 1030. Athletic directors and coaches are provided with said training on the infection, how to identify said infection, the risks of spreading the infection and methods of reducing the spread of infections. Similarly in child care and elder care situations, administrators and caregivers are provided with said education and training.

Although particular embodiments of the invention have been described in detail herein with reference to the accompanying drawings, it is to be understood that the invention is not limited to those particular embodiments, and that various changes and modifications, including the omission of steps or the inchangability of the order of steps, may be effected therein by one skilled in the art without departing from the scope or spirit of the invention.

What is claimed:

1. A method for controlling the spread of bacterial infections that colonize in the nose comprising:
identifying a group of individuals who are carriers, or at risk of carrying, or becoming carriers of a bacteria that colonizes in the nose,
providing a plurality of antibiotic strips capable of transdermal delivery of an antimicrobial agent from the strip to the nose, and
applying the plurality of strips to the noses of a plurality of individuals identified as the group who are carriers, or at risk of carrying, or becoming carriers of a bacterial infection that colonizes in the nose.

2. The method of claim 1, wherein the group of individuals is identified by recognizing a known likelihood of incidence of a bacterial infection that colonizes in the nose, and recognizing a need or desire to reduce the risk of spreading the bacterial infection.

3. The method of claim 1, wherein the group of individuals are doctors, patients, staff, and/or visitors of hospitals, skilled nursing facilities, other health care institutions.

4. The method of claim 1, wherein the group of individuals are identified by determining whether said group of individuals are present at locations where people come in close proximity to each other.

5. The method of claim 4, wherein the locations are schools, dorms, military barracks, camps, and/or areas of confinement including trains, plains, subways.

6. The method of claim 1, wherein the group of individuals are identified by conducting and/or examining the results of a study concerning incidences of propagation of bacterial infections among a population.

7. The method of claim 1, wherein the group of individuals are identified by examining reports showing populations, activities, and/or locations that have a high incidence of infection resulting from colonization of the noses of carriers.

8. The method of claim 1, wherein the group of individuals are identified by taking cultures of one or more individuals to determine whether said one or more individuals are infected and/or are carriers of a targeted bacterial infection.

9. A method of reducing the risk of infection of wounds, including surgical wounds, comprising the steps of:
identifying a preoperative care plan for a patient,
including as part of the preoperative care plan, the steps of providing an antibiotic strip capable of transdermal delivery of an antimicrobial agent from the strip to the nose, and
applying the antibiotic strip to the nose of the patient.

10. The method of claim 9, further including the step of screening the patient for a bacterial infection that colonizes in the nose.

11. The method of claim 9, wherein the step of applying the antibiotic strip occurs prior to the patient undergoing a surgical procedure.

12. The method of claim 9, further including the step of releasing an antimicrobial agent from the antibiotic strip into the nose of the patient.

13. The method of claim 9, further including the step of eradicating or partially eradicating a bacteria contained within or located near the nose of the patient.

14. The method of claim 13, wherein the bacteria is MRSA, and the antimicrobial agent contains silver.

15. A method of reducing the spread of bacterial infections at a location or facility and/or associated with an activity comprising the steps of:
classifying one or more individuals as targets for treatment,
providing a plurality of antibiotic strips capable of transdermal delivery of an antimicrobial agent from the strip to the nose,
treating one or more individuals by causing the application of one or more of the plurality of antibiotic strips to the noses of the one or more individuals, and
eradicating or partially eradicating a colony of bacteria contained within or located near the nose of the one or more individuals treated.

16. The method of claim 15, further including the step of causing a release of an antimicrobial agent from one or more of the plurality of antibiotic strips to the nose of the one or more individuals.

17. The method of claim 1, further including the step of providing administrators or other responsible individuals with education and training information, relating to infection control, to assist the administrators or other responsible people with identifying infection outbreaks, and infection risks, and undertaking a regimen to reduce the spread of bacterial infections.

18. The method of claim 15, further including the step of providing administrators or other responsible individuals with education and training information, relating to infection control, to assist the administrators or other responsible people with identifying infection outbreaks, and infection risks, and undertaking a regimen to reduce the spread of bacterial infections.

19. The method of claim 15, wherein the colony of bacteria comprises MRSA.

20. The method of claim 15, wherein the antimicrobial agent comprises silver.
